(19) United States
(12) Patent Application Publication
(10) Pub. No.: US 2009/0258782 A1
(43) Pub. Date: Oct. 15, 2009

(54) MESOPOROUS CARBONS

(75) Inventors: Yury Gogotsi, Warminster, PA (US); Gleb Yushin, Atlanta, GA (US); Sergey Victorvich Mikhalovsky, Brighton (GB); Andrew William Lloyd, East Sussex (GB); Gary James Phillips, East Sussex (GB)

Correspondence Address: WOODCOCK WASHBURN LLP, CIRA CENTRE, 12TH FLOOR, 2929 ARCH STREET, PHILADELPHIA, PA, 19104-2891, US

(73) Assignee: Drexel University, Philadelphia, PA (US)

(21) Appl. No.: 12/096,526

(22) PCT Filed: Dec. 8, 2006
(86) PCT No.: PCT/US06/47129

§ 371 Date: Mar. 18, 2009

Related U.S. Application Data
(60) Provisional application No. 60/749,117, filed on Dec. 9, 2005.
(60) Provisional application No. 60/835,644, filed on Aug. 4, 2006.

(51) Int. Cl. C01B 31/08; B01J 20/26; B32B 3/26

(52) U.S. Cl. 502/402; 502/414; 502/416; 428/304.4

(57) ABSTRACT

Provided are products, systems, and methods relating to the removal of particles from fluid samples using mesoporous carbon materials.

### CROSS-REFERENCE TO RELATED APPLICATIONS

This application claims benefit of U.S. Provisional Application No. 60/749,117, filed Dec. 9, 2005, and U.S. Provisional Application No. 60/835,644, filed Aug. 4, 2006, the disclosures of which are hereby incorporated by reference in their entirety.

### FIELD OF THE INVENTION

Provided are products, systems, and methods relating to the removal of particles from fluids using carbon-based materials.

### BACKGROUND OF THE INVENTION

There exists great interest among biomedical practitioners in improved products and methods for the removal of toxins, wastes, and other undesired molecules from fluids, including biofluids. For example, reducing the presence of inflammatory proteins from the blood of a subject enduring sepsis or an autoimmune condition can constitute life-saving therapy.

Sepsis is characterized by a systemic inflammatory response to bacterial infection. With over 18 million cases recorded annually worldwide and the absence of efficient sepsis drugs, this disease is a leading cause of death. Severe sepsis constitutes 17% of documented sepsis cases, has a current mortality rate 30-40% and globally kills more than 1,500 people every day. The rate of mortality caused by severe sepsis therefore occurs on a scale comparable to lung and breast cancer (˜2,700 and ˜1,100 people/day, respectively), leukemia (˜700 people/day), and AIDS (˜8,500 people/day). From an economic perspective, sepsis places a significant burden on the healthcare system, with the cost of treatment in the U.S. alone totaling over $17 billion. Angus D C et al. Critical Care Medicine, 2001. 29(7): 1303-1310.

The inflammatory response to various bodily insults is driven by the complex network of inflammatory mediators, mainly proteins called cytokines. See Asachenkov A et al. IEEE Trans. Biomed. Eng., 1994. 41: 943-953; Callard R et al. Immunity, 1999. 11: 507-513; Neugebauer E et al. Shock, 2001. 16: 252-258. In order to alleviate the inflammatory state of sepsis, for example, cytokines can removed from a subject's blood. Therapies aimed at simultaneous reduction of cytokines across the wide range of molecular sizes may prove more effective than drugs directed against some single inflammatory mediators. Asachenkov A et al.; Callard R et al.; Natanson C et al. Crit. Care Med., 1998. 26: 1927-1931.

Hemofiltration or hemoadsorption could allow extracorporeal removal of inflammatory cytokines in an amount that is sufficient to decrease the inflammatory response. While both sieving and adsorption could play a role in hemofiltration, the adsorption characteristics of the filter material are generally believed to be a dominant factor in membrane efficiency. Additionally, adsorption can remove toxins without introducing any other substances into the blood. The use of hemoadsorption during hemofiltration in that hemoadsorption could have the same or enhanced efficiency in the treatment of autoimmune diseases or other conditions resulting in an inflammatory response, could be of lower cost, and may offer considerably better comfort for patients during and after the treatments.

Porous carbons may be used for the purification of various biofluids. Activated carbons (“ACs”) have been known for over three thousand years and still remain the most powerful conventional adsorbents (see Mikhalovsky S V. Perfusion-UK, 2003. 18:47-54), mainly due to their highly developed porous structure and large surface area. Most of the specially purified activated carbons that are prepared from synthetic polymers show excellent biocompatibility, and do not require special coatings for direct contact with blood. S V Mikhalovsky S V; Sandeman S R et al. Biomaterials, 2005. 26(34):7124-7131. However, despite extensive studies and improvements in activation processes, little control over the pore structure has been achieved. Even advanced ACs show partial performance in adsorbing large inflammatory proteins, mostly due to a limited surface area accessible to the adsorbate. Templating has been used to increase the volume of larger pores. Ryoo R et al. J. Phys. Chem. B, 1999. 103(37): 7743-7746; Xia Y D & Mokaya R. Advanced Mater., 2004. 16(11):886-891; Lee J et al. J. Mater. Chem., 2004. 14(4): 478-486. Porous carbon has been prepared by introducing carbon into the pores of alumina or silica, followed by removal of the oxide template by acidic treatment. Apart from the high cost of performing such techniques, the resulting carbon exhibits poor mechanical integrity and near-spherical pore shape. Furthermore, pore bottlenecks prevent the adsorption of large molecules into the carbon particles, and therefore only a relatively small external surface area is available for adsorption. Small particles (<100 nm in diameter) would offer a larger external surface area, but cannot be used in most relevant biomedical applications due to the difficulty of filtering such particles from biofluids in which they are used. The pore size in other porous carbon materials such as carbon nanotubes (“CNTs”) is very difficult to control or tune to the desired value. Most CNTs have low specific surface area (“SSA”), and agglomeration of CNTs into ropes, which frequently occurs when CNTs are brought into contact with biofluids, further significantly reduces their accessible surface area.

Carbon produced by etching of one or more metals from metal carbides, called carbide-derived carbon (“CDC”), has been recently shown to offer a great potential for controlling the size of micropores, which typically range from 0.4 to 2 nm in diameter. Y Gogotsi et al. Nature Materials, 2003. 2:591-594. Known CDCs are generally produced by chlorination of carbides in the 200-1200° C. temperature range. Metals and metalloids are removed as chlorides, leaving behind a collapsed noncrystalline carbon with up to 80% open pore volume. The detailed nature of the porosity—average size and size distribution, shape, and total specific surface area (“SSA”)—can be tuned with high sensitivity by selection of precursor carbide (composition, lattice type) (see id.; R. K Dash et al., Microporous and Mesoporous Materials, 2004. 72: p. 203-208; R. K Dash, G. Yushin, G. Laudisio, J. E. Fischer, and Y. Gogotsi, Synthesis and Characterization of Nanoporous Carbon Derived from Titanium Carbide. Carbon, submitted, 2006; R. K Dash, G. Yushin, and Y Gogotsi, Synthesis, Structure and Porosity Analysis of Microporous and Mesoporous Carbon Derived from Zirconium Carbide. Microporous and Mesoporous Materials, in press, 2005) and chlorination temperature (Y Gogotsi et al.). As yet, however, only tuning of microporosity, but not of pores having larger diameters, has been demonstrated in CDC.

### SUMMARY OF THE INVENTION

Disclosed are porous carbons that can have controlled volume, size, and surface area characteristics. The inventive carbons can be prepared using novel CDC synthesis from selected ternary (MAX-phase) carbides as starting materials. Also provided are novel systems for the adsorption of particles from fluids, methods for producing porous carbons, as well as methods for the removal of particles from fluids.

One aspect of the present invention provides carbon compositions that are useful in adsorbing particles from fluids. In one embodiment there are provided carbon compositions produced from a carbon-containing inorganic precursor comprising a plurality of pores, a plurality of said pores having characteristic dimensions from about 4 to about 50, wherein said compositions adsorb one or more particles from a fluid.

Another aspect of the present invention comprises adsorption systems comprising carbide-derived carbon compositions. In one embodiment there are provided adsorption systems comprising carbon compositions produced from a carbon-containing inorganic precursor comprising a plurality of pores, a plurality of said pores having characteristic dimensions from about 4 to about 50, wherein said compositions adsorb one or more particles from a fluid.

A further aspect of the present invention comprises methods for adsorbing particles from a fluid that contains particles. In one embodiment, there are provided methods of adsorbing particles from a fluid having particles comprising contacting said fluid with a carbon composition produced from a carbon-containing inorganic precursor comprising a plurality of pores, a plurality of said pores having characteristic dimensions from about 4 to about 50.

In an additional aspect of the present invention there are provided methods for making carbide-derived carbon compositions. In one embodiment there are disclosed methods of making a carbide-derived carbon composition comprising heating a ternary carbide sample, and, during said heating, chlorinating said ternary carbide sample. Also provided are carbide-derived carbon compositions produced according to the disclosed methods.

### BRIEF DESCRIPTION OF THE DRAWINGS

The summary, as well as the following detailed description, is further understood when read in conjunction with the appended figures. For the purpose of illustrating the invention, there are shown in the figures exemplary embodiments of the invention; however, the invention is not limited to the specific methods, compositions, characteristics, and devices disclosed.

FIG. 1 illustrates two schematics of protein adsorption by porous carbons.

FIG. 2 depicts N₂ sorption isotherms for the inventive and commercially available carbon samples.

FIG. 3 provides a graphical depiction of the distribution of pore sizes of porous carbons in the 1.5 to 36 nm range obtained from N₂ sorption isotherms.

FIG. 4 provides a graphical depiction of the distribution of pore sizes of porous carbons in the 0.4 to 4 nm range obtained from Ar sorption isotherms.

FIG. 5 provides images from transmission electron microscopy of porous carbon samples.

FIG. 6 is a comparison of the efficiencies of the inventive and commercially available carbon samples with regard to the removal of cytokines from human blood plasma.

FIG. 7 depicts the results of measurements of the adsorption of cytokines by porous carbons as a function of accessible surface area.

### DETAILED DESCRIPTION OF ILLUSTRATIVE EMBODIMENTS

The present invention may be understood more readily by reference to the following detailed description taken in connection with the accompanying figures and examples, which form a part of this disclosure. It is to be understood that this invention is not limited to the specific products, methods, conditions or parameters described and/or shown herein, and that the terminology used herein is for the purpose of describing particular embodiments by way of example only and is not intended to be limiting of the claimed invention.

In the present disclosure the singular forms “a,” “an,” and “the” include the plural reference, and reference to a particular numerical value includes at least that particular value, unless the context clearly indicates otherwise. Thus, for example, a reference to “a carbon-containing inorganic precursor” is a reference to one or more of such precursors and equivalents thereof known to those skilled in the art, and so forth. When values are expressed as approximations, by use of the antecedent “about,” it will be understood that the particular value forms another embodiment. Where present, all ranges are inclusive and combinable.

Provided are porous carbons that can be used for the efficient removal of particles from fluids. For example, the present carbons can be used for the removal from blood or other biofluids of bioparticles such as inflammatory mediators or other large organic molecules, viruses, or other “large” molecules or particles. The disclosed carbons can be generally characterized as having pores with tunable volume and surface area attributes, and display high-efficiency adsorption of particles from fluids with which they are contacted. The efficiency of the removal of particles from fluids by the present carbide-derived carbons provides results that are comparable to those that employ highly-specific antibody-antigen interactions. The detailed nature of porosity in carbons—such as average size and size distribution, shape, volume, and specific surface area (“SSA”)—can be tuned with high sensitivity by manipulating such factors as the choice of precursor carbide and chlorination temperature (see, e.g., Gogotsi Y et al. Nature Materials, 2003. 2:591-594), yet only tuning of microporosity (characterized by pores having diameters in the range of 0.4 to 2 nm) has been demonstrated in carbide derived carbons. In contrast, the instant carbons can evince mesopores (pores having diameters above 2 nm up to about 50 nm) with tunable pore size, volume, and surface area characteristics, which are important definers of particle adsorption aptitude.

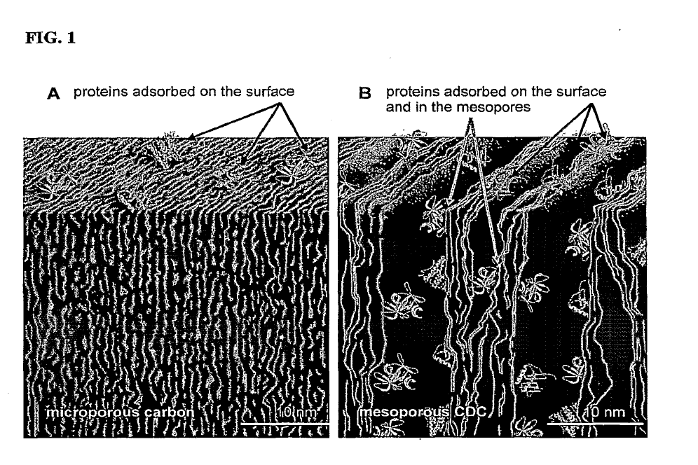

Synthesis of the disclosed carbons can be accomplished by selecting carbon-containing inorganic precursors as starting materials. Such carbon-containing inorganic precursors can include carbonitrides or carbides, such as commercially available carbide-derived carbons (“CDCs”), as starting materials. The starting materials can also comprise ternary carbonitrides or ternary carbides. The ternary carbides can be from the MAX phase group of layered carbides. For example, commercially available powders from the MAX-phase group of ternary carbides, such as Ti₂AlC and Ti₃AlC₂ (available from 3ONE2, Inc., Voorhees, N.J.), can be utilized to produce the inventive carbons. Although starting materials from the MAX-phase group of ternary carbides are preferred, other suitable carbide starting materials, which are readily determined by those skilled in the art, may be selected according to the particular needs of the manufacturer. Example 1, infra, describes an exemplary process for the synthesis of the disclosed carbons. Slit-shaped open pores are characteristically observed in CDCs produced from the Ti₂AlC and Ti₃AlC₂ carbides (see Gogotsi Y et al. Nature Materials, 2003. 2:591-594; Yushkin G et al. Carbon, 2005. 44(10): 2075-2082; Hoffman E et al. Chem. Mater., 2005. 17(9): p. 2317-2322), and the instant mesoporous CDCs can evince such slit-shaped pores. FIG. 1 illustrates the schematics of particle adsorption by porous carbons having microporous and slit-shaped mesoporous surface profiles, demonstrating the mechanics of superior “large” particle adsorption by mesoporous carbons. The present carbons therefore represent a highly advantageous means for the selective optimized adsorption of a wide variety particles, including biomolecules, from fluids such as biofluids. As used herein, “biofluids” is meant to include biological fluids such as, but not limited to, blood, serum, plasma, urine, saliva, and cerebral spinal fluid. Biofluids also encompasses fluids used in biological processes such as cell culturing, fermentation, and the like.

Accordingly, there are provided carbon compositions produced from a carbon-containing inorganic precursor comprising a plurality of pores, a plurality of said pores having characteristic dimensions from about 4 to about 50 μm, wherein said composition adsorbs one or more particles from a fluid. A substantial proportion of the pores can be substantially slit shaped. As used herein, a “substantial proportion” means a non-rare occurrence thereof. Because pores may be present in numerous configurations, including, inter alia, substantially cylindrical or substantially slit-shaped, or otherwise, the term “characteristic dimensions” is used herein to describe diameter in the case of substantially cylindrical pores, and to describe width in the case of substantially slit-shaped pores. In some embodiments, the disclosed carbon compositions have a total pore volume greater than 1.27 cc/g, as measured by N₂ adsorption at 77 K (i.e., at 77 kelvins). The disclosed carbons can comprise a plurality of pores having characteristic dimensions greater than about 4 nm, wherein the total volume of pores having characteristic dimensions greater than about 4 nm is greater than 0.554 cc/g, as measured by N₂ adsorption at 77 K. Carbons having pores with characteristic dimensions exceeding about 4 nm are useful for adsorption of particles having one or more physical dimensions less than or equal to about 4 nm, such as the interleukin-8 cytokine, an inflammatory protein that has been measured as having dimensions of 4×4×9 nm. Rajarathnam K et al. Biochemistry, 1995. 34(40):12983-12990. The disclosed carbons can also comprise a plurality of pores having characteristic dimensions greater than about 5 nm, wherein the total volume of said pores having characteristic dimensions greater than about 5 nm is greater than 0.434 cc/g, as measured by N₂ or Ar adsorption and analyzed according to the Brunauer-Emmet-Teller method. Particles such as the interleukin-6 cytokine (dimensions 5×5×12.2 nm; see Somers W et al. Embo Journal, 1997. 16(5):989-997) are therefore readily adsorbed from fluids by these carbons. The provided carbons can likewise comprise a plurality of pores having characteristic dimensions greater than about 5.5 nm, wherein the total volume of said pores having characteristic dimensions greater than about 5.5 nm is greater than 0.377 cc/g, as measured by N₂ or Ar adsorption and analyzed according to the non-local density functional theory method. Interleukin-1β (dimensions 5.5×5.5×7.7 nm; see Einspahr H et al. J. Cryst. Growth, 1988. 90(1-3):180-187) and other particles having dimensions less than about 5.5 nm can be removed from fluids using these carbons. Carbons comprising a plurality of pores having characteristic dimensions greater than about 9.5 nm, wherein the total volume of said pores having characteristic dimensions greater than about 9.5 nm is greater than 0.0824 cc/g, as measured by N₂ adsorption at 77 K, are also provided herein. The well-known cytokine TNF-α (9.4×9.4×11.7 nm trimer dimensions; Reed C et al. Protein Engineering, 1997. 10(10):1101-1107) and other particles having dimensions less than about 9.5 nm can be adsorbed from fluids using the disclosed carbons.

Sorption isotherms can be used to measure surface area and volume characteristics, and may be analyzed using an number of methodologies. The Brunauer-Emmet-Teller (BET) method and non-local density functional theory (NLDFT) method can be used to reveal the specific surface area and pores size distributions (PSD) of carbide derived carbons. Gregg S J & Sing K S W, 1982, London: Academic Press. 42-54; Ravikovitch P I & Neimark A V, Colloids and Surfaces, 2001. 187-188:11-21; Brunauer S et al. J. of American Chemical Society, 1938. 60: 309-319; Lowell S & Schields J E. Powder Surface Area and Porosity. Chapman & Hall. 1998, New York. 17-29. For example, disclosed are carbon compositions having a total specific surface area greater than 1652 m²/g, as measured according to the Brunauer-Emmet-Teller method. There are also provided carbon compositions having a total specific surface area greater than 1362 m²/g, as measured using N₂ or Ar adsorption and analyzed according to the non-local density functional theory method. The present carbons can have a N₂ sorption profile of at least 1000 cc/g N₂ at 1.0 P/Pₒ (relative pressure).

There are also provided carbon compositions produced from a carbon-containing inorganic precursor comprising a plurality of pores having characteristic dimensions from about 4 and up to about 50 nm, said pores having a total specific surface area greater than 172 m²/g, as measured by N₂ adsorption at 77 K. The carbon compositions can comprise particles of Ti₂AlC reacted with chlorine at or exceeding a temperature of about 600° C., 800° C., or 1200° C. The carbon compositions can also comprise particles of Ti₃AlC₂ reacted with chlorine at or exceeding a temperature of about 600° C., 800° C., or 1200° C.

The present carbon compositions are capable of efficient adsorption of particles from fluids, including biofluids. The particles can be one or more proteins, and the proteins may be inflammatory mediators, which include cytokines. For example, the disclosed carbon compositions can permit adsorption of the TNF-α, IL-1β, IL-8, or IL-6 cytokines from a fluid, such as a human plasma sample. In some embodiments, the disclosed compositions are capable of adsorbing at least about 40%, at least about 60%, or at least about 80% of TNF-α from a fluid sample in about 60 min. The CDCs can also adsorb at least about 50%, at least about 70%, or at least about 90% of IL-6 from a fluid sample in about 60 min. The adsorption efficiency of the present carbon compositions of course depends on the amount of carbon composition that is used relative to the particle-containing fluid. Thus, an adsorption mixture containing 50 mg carbon composition per milliliter of fluid will display a higher adsorption efficiency than a 20 mg/ml mixture. The scope of the present invention is intended to include any carbon composition that is capable of adsorbing particles from a fluid when used at any concentration.

The specific surface area of a porous carbon is one descriptor of the carbon's adsorption characteristics, and it is widely appreciated that higher specific surface areas are more highly desirable. Specific surface area can be measured in terms of the total specific surface area of a given mass of material (i.e., including pores of all sizes), or may be measured according to the aggregated specific surface area only of those pores having characteristic dimensions that exceed a particular measurement. The latter type of specific surface area measurement is particularly instructive in the context of those applications wherein a particle having known dimensions represents the adsorption target; during such applications, only the specific surface area of those pores that have characteristic dimensions that equal or exceed the dimensions of the adsorption target is relevant to the determination of the adsorption characteristics of the porous carbon. There are provided carbon compositions produced from a carbon-containing precursor comprising a plurality of pores, a plurality of said pores having characteristic dimensions greater than 5 nm, said pores with characteristic dimensions greater than 5 nm having a total specific surface area greater than 120 m²/g, as measured by N₂ adsorption at 77 K. The adsorption of particles having dimensions less than about 5 nm are therefore implicated by these carbons. The disclosed compositions can also comprise a plurality of pores having characteristic dimensions greater than 5.5 nm, said pores with characteristic dimensions greater than 5.5 nm having a total specific surface area greater than 98.3 m²/g, as measured by N₂ adsorption at 77 K. Particles having dimensions less than about 5.5 nm are readily adsorbed by these carbons. Also provided are carbon compositions comprising a plurality of pores having characteristic dimensions greater than 9.5 nm, said pores with characteristic dimensions greater than 9.5 nm having a total specific surface area greater than 14.6 m²/g, as measured by N₂ adsorption at 77 K. Larger particles, such as the TNF-α cytokine trimer (9.4×9.4×11.7 nm) can be adsorbed thereby.

Also disclosed are carbon compositions produced from a carbon-containing inorganic precursor comprising a plurality of pores, at least about 30 volumetric percentage of said pores, as measured by N₂ adsorption at 77 K, having characteristic dimensions equal to or greater than about 9.5 nm, wherein said carbon composition adsorbs TNF-α from a fluid. As used herein, “volumetric percentage” means the percentage of total pore volume that is attributable to those pores having the specified characteristic dimensions. In other embodiments, at least about 50 or at least about 70 volumetric percentage of said pores, as measured by N₂ adsorption at 77 K, have characteristic dimensions equal to or greater than about 9.5 nm. In other disclosed carbon compositions comprising a plurality of pores, at least about 30, at least about 50, or at least about 70 volumetric percentage of said pores, as measured by N₂ adsorption at 77 K, have characteristic dimensions greater than about 5.5 nm, and such carbon compositions adsorb IL-1β from a fluid. Also provided are carbon compositions comprising a plurality of pores in which at least about 30, at least about 50, or at least about 70 volumetric percentage of said pores, as measured by N₂ adsorption at 77 K, have characteristic dimensions greater than about 5 nm, and such carbon compositions adsorb IL-6 from a fluid. The characteristics of the present carbon compositions comprising a plurality of pores can also be such that at least about 30, at least about 50, or at least about 70 volumetric percentage of said pores, as measured by N₂ adsorption at 77 K, have characteristic dimensions greater than about 4 nm, and such carbon compositions adsorb IL-8 from a fluid.

In their manufactured state, present carbon compositions typically comprise a substantially granular or particulate conformation, such as a powder. For some applications, it may be advantageous for the inventive carbons to be available in a substantially non-particulate form, such as a form in which the individual carbon composition particles are bound to one another. In such a form, the carbon composition can be easily manipulated, and even molded into a desired configuration, for example, a cylinder for incorporation into a filtration apparatus. Accordingly the present carbon compositions may further comprise a binder that enables the adhesion of composition particles to one another. Such binders preferably comprise polymers, many types of which are readily identified by those skilled in the art, but may comprise any material that functions to join composition particles to one another and that does not substantially interfere with the adsorption activity of the disclosed carbons. An exemplary binder polymer is teflon. When the instant carbon compositions are intended for the adsorption of particles from a biofluid, the selected binder is preferably compatible with such a use in terms of medical safety and efficacy.

The inventive carbons can be used in the construction of novel adsorption systems for the efficient removal particles from fluids. Such adsorption systems represent low cost, high comfort, optimized means for such applications as hemoadsorption for the removal of such bioparticles as toxins or inflammatory cytokines. Because they may incorporate any of the disclosed carbon compositions, the adsorption characteristics of such systems can be described according to the detailed, tunable nature of the porosity of the inventive carbon compositions, including average size and size distribution, shape, volume, and specific surface area. Thus, there are also provided adsorption systems that include any of the inventive carbon compositions as previously disclosed, or any combination thereof.

Methods for the adsorption of particles from a fluid having particles are also enabled through use of the inventive carbons. The provided methods comprise contacting a fluid having particles with any of the previously disclosed carbon compositions, or any combination thereof. The present methods employ the inventive carbons and the specific, tunable porosity by which they are characterized, permit the highly efficient, selective sorption of a wide variety of particles from fluids, and can therefore be advantageously used with broad array of medical, biochemical, or industrial applications.

The detailed, distinctive porosity and adsorption characteristics of the disclosed carbons are made possible through specialized, previously unknown production methods that use carbon-containing inorganic precursors as starting materials. Disclosed are novel methods of making a carbon composition having pores, at least 40% of said pres having characteristic dimensions from about 5 to about 50 nm, such methods comprising heating a carbon-containing inorganic precursor; and, during said heating, halogenating the inorganic precursor. Chlorination can be used for such halogenating. The carbon-containing inorganic precursor may be a ternary carbide. Exemplary ternary carbides include Ti₂AlC, Ti₃AlC₂, or any other suitable ternary carbide. The heating can occur at or can exceed 600° C., 800° C., 1000° C., or 1200° C. The heating can occur in a furnace, and the method can include purging the furnace prior to the heating of the inorganic precursor. The purging of the furnace is preferably performed for 30 minutes, but other durations, whether longer are shorter, can be acceptable. Purging with a gas that is inert relative to carbon is preferred, with noble gases being more highly preferred, one exemplary embodiment employing Ar as the purging material.

The halogenation period, during which gaseous halogen, such as chlorine (Cl₂), flows over the heated inorganic precursor, can be performed for about 3 hours, at a flow rate of about 10 sccm. The duration of the halogenation and the flow rate at which the halogen flows into the furnace depend upon the quantity of precursor that is used. Accordingly, the halogenation period and flow rate may range as broadly as is necessitated by the quantity of precursor that is present.

After the halogenation process has proceeded to completion, the chlorinated ternary carbide sample may be cooled. Such cooling can persist for up to 5 hours, or can be extended beyond that length of time, cooling for about 5 hours being preferred. A flow of gas across the carbide sample can be used during the cooling process, and a noble gas such as Ar may be used for this purpose. A cooling gas flow rate of 40 sccm of Ar represents one exemplary embodiment. The cooling gas can be removed during the cooling process, and an exhaust tube can be used for this purpose.

The present methods, which can be practiced using any combination of the disclosed parameters, therefore permit the synthesis of specialized carbons. Porous carbons produced according to the inventive methods are also within the scope of the instant invention.

The present invention is further defined in the Examples included herein. It should be understood that these examples, while indicating preferred embodiments of the invention, are given by way of illustration only, and should not be construed as limiting the appended claims From the present disclosure and these examples, one skilled in the art can ascertain the essential characteristics of this invention, and without departing from the spirit and scope thereof, can make various changes and modifications of the invention to adapt it to various usages and conditions.

### Example 1

### Synthesis of Carbide-Derived Carbons

CDCs were synthesized from Ti₂AlC and Ti₃AlC₂ powders by the reaction with pure chlorine (99.5%, BOC gases) at 600, 800 and 1200° C. Both carbides were produced at Drexel University, but are now commercially available (3-ONE-2, Inc, NJ, US). The Ti₂AlC and Ti₃AlC₂ carbides belong to the MAX-phase group of ternary carbides, having a layered hexagonal structure with carbon atoms positioned in basal planes and separated by 0.68 nm (Ti₂AlC) or alternating layers of 0.31 and 0.67 nm (Ti₃AlC₂). Barsoum M W. Chemistry. 2000; 28:201-81. The CDCs produced from these carbides are known to posses slit-shaped open pores Gogotsi Y et al. Nature Materials. 2003; 2:591-4; Yushin G et al. Carbon. 2005 44(10):2075-82; Hoffman E et al. Chem Mater. 2005; 17(9):2317-22. The average particle size of the carbide samples used in the present experiments was ˜10 μm, as measured using a particle size analyzer (Horiba LA-910, Japan). For CDC synthesis, the selected carbide powder was placed onto a quartz sample holder and loaded into the hot zone of a horizontal quartz tube furnace. Prior to heating, the tube (˜30 mm in diameter) was purged with high purity Ar (BOC Gases, 99.998%) for 30 minutes at a flow rate of 100 sccm. Once the desired temperature was reached and stabilized, the Ar flow was stopped and a 3-hour chlorination began with Cl₂ flowing at a rate of 10 sccm. After the completion of the chlorination process, the samples were cooled down under a flow of Ar (40 sccm) for about five hours to remove any residual chlorine or metal chlorides from the pores, and taken out for further analyses. In order to avoid a back-stream of air, the exhaust tube was connected to a bubbler filled with sulphuric acid. A detailed description of the chlorination apparatus used in this study can be found at Yushin G, Gogotsi Y, Nikitin A. Carbide Derived Carbon. In: Gogotsi Y, editor. Nanomaterials Handbook, Boca Raton, Fla.: CRC Press; 2005. p. 239-82.

### Example 2

### Characterization

The sorption performance of the CDCs was compared with that of Adsorba 300C and CXV carbon adsorbents. Adsorba 300C (NORIT Americas, Inc., Marshall, Tex.) is an activated carbon produced from peat, and coated with a 3-5 μm thick cellulose membrane for better hemocompatibility. It is commercially used in adsorbent-assisted extracorporeal systems manufactured by Gambro, Sweden. CXV is an activated carbon obtained from CECA (subsidiary of Arkema, Inc., Paris, France), known to be extremely efficient for cytokine removal applications and thus used as a benchmark reference.

The structure of the CDCs was investigated using high-resolution transmission electron microscopy (HRTEM). The TEM samples were prepared by two minutes sonication of the CDC powder in isopropanol and deposition on the lacey-carbon coated copper grid (200 mesh). A field-emission TEM (JEOL 2010F, Japan) with an imaging filter (Gatan GIF) was used at 200 kV.

The porosity of the produced CDCs was studied using automated micropore gas analyzers Autosorb-1 and Nova (Quantachrome Instruments, Boynton Beach, Fla.). N₂ and Ar sorption isotherms were obtained at liquid nitrogen temperature (−196° C.) in the relative pressure P/P₀ range of about 8×10⁻⁷ to 1 and 2×10⁻² to 1, respectively. The isotherms were analyzed using Brunauer-Emmet-Teller (BET) equation and non-local density functional theory (NLDFT) to reveal the specific surface area (SSA) and pore-size distributions (PSD) of the CDCs. The SSAs calculated using BET and DFT theory are referred to as BET-SSA and DFT-SSA, respectively. A difference in absolute values between BET-SSA and DFT-SSA is expected, as both types of calculations are based on different assumptions, which might not be justified with the utmost accuracy for all the materials under study. Quantachrome Instruments data reduction software Autosorb v.1.50 (Ravikovitch P I & Neimark A V. Colloids and Surfaces A: Physicochemical and Engineering Aspects. 2001; 187-188:11-21) was employed for the porosity analysis. Slit-shaped pores were assumed for the calculations.

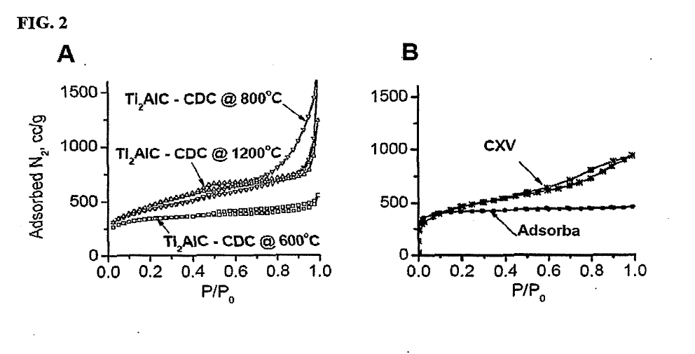

FIG. 2 shows the N₂ sorption isotherms of CDCs (FIG. 2A) and commercial carbon samples (FIG. 2B). All the samples, except Adsorba 300C, demonstrate type IV isotherm according to the Brunauer classification (Gregg S J & Sing K S W. Adsorption, Surface Area and Porosity. London: Academic Press; 1982) with a characteristic hysteresis, suggesting the presence of mesopores (pores with size in the 2-50 nm range). CDCs from both Ti₂AlC (FIG. 2A) and Ti₃AlC₂ (not shown) demonstrate similar trends as the temperature of synthesis changes. The volume of N₂ adsorbed in the porous structure of CDC prepared at lower temperature (600° C.) approaches half of the maximum values at low relative pressure (P/P₀). The steep slope of the adsorption curve at P/P₀ values approaching 1, associated with capillary condensation in mesopores, is quite short, suggesting a small mesopore volume. Ravikovitch P I & Neimark A V Colloids and Surfaces A: Physicochemical and Engineering Aspects. 2001; 187-188:11-21. The N₂ sorption behavior changes dramatically at intermediate (˜800° C.) chlorination temperatures. The total volume of adsorbed N₂ more than doubles; an increase is observed over the whole P/P₀ range, indicating a significant increase in both the total and mesopore volume. The level of adsorption-desorption hysteresis, and the steep slope as P/P₀ approaches unity also increases substantially, in agreement with the suggested increase in the relative volume of mesopores. As the synthesis temperature increases to 1200° C., the volume of adsorbed N₂ further increases in the P/P₀ range of up to ˜0.8, but becomes lower at higher P/P₀ values (FIG. 2A). Such changes in the isotherm shape indicate the reduction in the relative volume of larger mesopores.

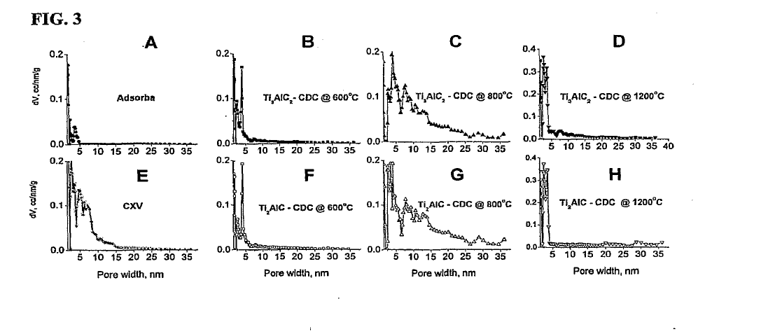

The pore size distribution (PSD) curves calculated in the 1.5-36 nm range for all the studied samples from the N₂ isotherms (FIG. 3) fully support the aforementioned conclusions. The CDC samples formed at low temperature (600° C.) have a very low volume of mesopores, particularly those above 10 nm (FIGS. 3B & 3F). At the intermediate synthesis temperatures (800° C.), the PSD becomes wider and shifts to higher pore-size values (FIGS. 3C & 3G). These samples clearly have the largest volume of mesopores above 5 nm. At the high chlorination temperature of 1200° C. the total CDC mesopore volume remains relatively high (FIGS. 3D & 3H). It is in fact higher than the total pore volume of many activated carbon samples, including Adsorba 300C (FIG. 3A). However, most of the mesopores in these samples are below 4-5 nm. Adsorba 300C has the smallest volume of mesopores and is almost purely microporous carbon. The PSD of the CXV sample (FIG. 3E) is close to that of an average of CDC samples produced at high and intermediate temperatures. The porosity data for all the studied samples are summarized in Table 1, below, in which results are presented with respect to the samples' surface area and pore volume accessible to the cytokines to be adsorbed. Such surface area and pore volume are approximated as the SSA and volume of pores exceeding the smallest protein dimension in size: 9.4 nm for TNF-α trimer, 5.5 nm for IL-1β, 5 nm for IL-6, and 4 nm for IL-8.

TABLE 1

| | | | Ti₃AlC₂- | Ti₃AlC₂- | Ti₃AlC₂- | Ti₂AlC- | Ti₂AlC- | Ti₂AlC- |
|---|---|---|---|---|---|---|---|---|
| | | | CDC, | CDC, | CDC, | CDC, | CDC, | CDC, |
| | Adsorba | CXV | 600° C. | 800° C. | 1200° C. | 600° C. | 800° C. | 1200° C. |
| BET-SSA, m²/g | 1589 | 1652 | 1285 | 920 | 1493 | 1348 | 1649 | 2100 |
| DFT-SSA, m²/g | 1362 | 1025 | 940 | 727 | 1037 | 1330 | 1412 | 1856 |
| SSA of pores above 9.5 | 0.76 | 14.6 | 8.53 | 98.2 | 30.7 | 12 | 95 | 48 |
| nm, m²/g | | | | | | | | |
| SSA of pores above 5.5 | 1.1 | 98.3 | 17.2 | 201 | 61.3 | 24.7 | 193 | 77 |
| nm, m²/g | | | | | | | | |
| SSA of pores above 5.0 | 1.12 | 120 | 20 | 223 | 67.6 | 29.1 | 224 | 88 |
| nm, m²/g | | | | | | | | |
| SSA of pores above 4.0 | 5.98 | 172 | 36.5 | 291 | 84.7 | 59.4 | 296 | 107 |
| nm, m²/g | | | | | | | | |
| Total pore volume, cc/g | 0.639 | 1.270 | 0.705 | 1.70 | 1.24 | 0.825 | 2.17 | 2.01 |
| Volume of pores above 9.5 | 0.0068 | 0.0824 | 0.074 | 0.781 | 0.257 | 0.104 | 0.817 | 0.497 |
| nm, cc/g | | | | | | | | |
| Volume of pores above 5.5 | 0.0081 | 0.377 | 0.105 | 1.163 | 0.370 | 0.152 | 1.2065 | 0.611 |
| nm, cc/g | | | | | | | | |
| Volume of pores above 5.0 | 0.0082 | 0.434 | 0.113 | 1.221 | 0.387 | 0.164 | 1.292 | 0.641 |
| nm, cc/g | | | | | | | | |
| Volume of pores above 4.0 | 0.0188 | 0.554 | 0.149 | 1.373 | 0.425 | 0.232 | 1.45 | 0.685 |
| nm, cc/g | | | | | | | | |

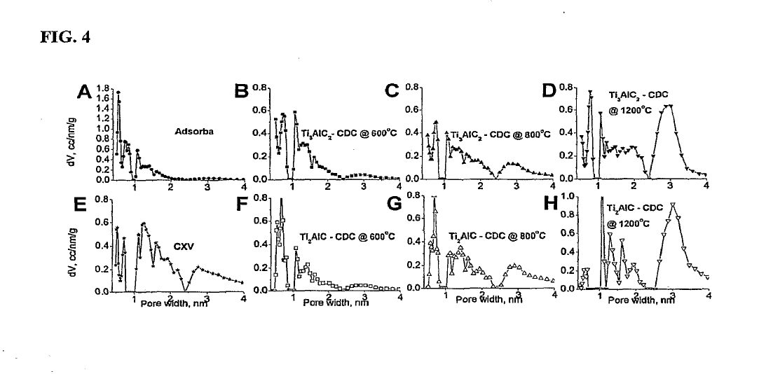

While Ar sorption is not a very efficient technique to study the large mesopores, it gives more accurate PSD results for small (<4 nm) pore values, mainly due to argon's smaller atomic size, the absence of quadrupole moment (which can potentially lead to localized adsorption as in case of N₂) and its weaker interactions with carbon adsorbents. The PSD of the studied samples in the 0.4-4 nm range obtained from Ar sorption isotherms (FIG. 4) revealed details of the samples' microporosity. Similar to Adsorba 300C, both CDC samples produced at 600° C. have the majority of pores below 2 nm in width. As the CDC synthesis temperature increases, the average size of the pores in the 0.4-4 nm range increases as well (FIG. 4). However, above 800° C., pores in the 2-4 nm range have a tendency to grow on the account of the micropores, forming a large volume of ˜3 mm pores at 1200° C. The PSD of the CXV sample is close to the average between the CDC samples formed at 800 and 1200° C.

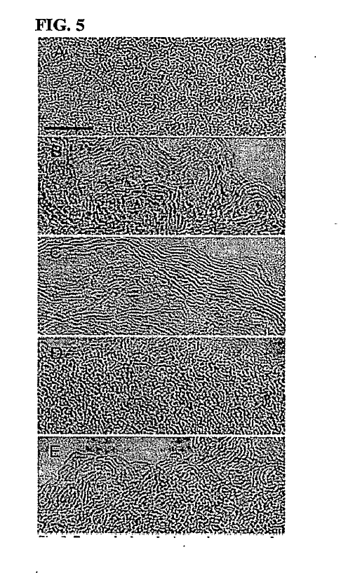

Characterization of microstructure by Transmission Electron Microscopy. Transmission electron microscopy (TEM) revealed disordered microstructure of all the studied carbons. The degree of disorder was different between the carbons. Both CDCs formed at 600° C. demonstrate completely amorphous structure, without any graphite fringes visible (FIG. 5A). Increasing the CDC processing temperature to 800° C. resulted in the formation of short curved graphene structures, considered turbostratic carbon (FIG. 5B). At 1200° C. TEM detected markedly increased ordering and the formation of long and thin (1-3 graphene sheets) graphite ribbons (FIG. 5C). At the edge of the particles, ribbons with up to 10 graphene layers were found (FIG. 5C). The microstructure of Adsorba 300C sample was found to be highly amorphous, (FIG. 5D) while that of the CXV carbon (FIG. 5E), turbostratic.

Previous studies have shown that the observed evolution of ordering within the carbon structure with the chlorination temperature is quite common for most of the CDCs obtained from both ternary and binary carbides. Yushin G, Gogotsi Y. Nikitin A. Carbide Derived Carbon. In: Gogotsi Y, editor. Nanomaterials Handbook. Boca Raton, Fla.: CRC Press; 2005. p. 239-82. The changes in the PSDs correlate to changes in the CDC microstructure. The low mobility of the carbon atoms at the low chlorination temperatures resulted in the formation of a uniform amorphous structure (FIG. 5A) with small micropores (FIGS. 3B, 3F, 4B, 4F). At higher synthesis temperatures, higher carbon mobility allowed for the formation of graphitic ribbon networks (FIG. 5C), with mesopores forming between the graphene ribbons and micropores in the imperfections of the graphitic ribbons or within the remaining disordered carbon (FIGS. 3D, 3H, 4D, 4H). At the intermediate temperature of 800° C., the mobility of carbon was high enough to allow for a redistribution of carbon atoms into defective graphene sheets and the collapse of several sheets into stacks forming mesopores between them. However, the mobility was still too low to allow uniform linking between the turbostratic ribbons, resulting in a wide distribution of mesopores (FIGS. 3C, 3G). Since precise determination of non-spherical pores in disordered non-planar particles is not possible using TEM, the present study relied on gas sorption measurements for the PSD determination.

### Example 3

### Particle Adsorption

Fresh frozen human plasma (NBS, UK) was defrosted and spiked with the recombinant human cytokines (TNF-α, IL-1β, IL-6, and IL-8; all obtained from BD Biosciences, San Jose, Calif.) at a concentration of about 1000, 500, 5000, and 500 pg/ml, respectively. These levels are comparable with the concentrations measured in the plasma of patients with sepsis. Cohen J & Abraham E. J Infect Dis. 1999; 180:116-21; Heering P et al. Int Care Med. 1997; 23:228-96; Marum S et al. Crit Care Med. 2000; 4:66. Carbon adsorbents (0.02 g) were equilibrated in phosphate buffered saline (PBS; 0.5 ml) overnight prior to removal of PBS and addition of 800 μl of spiked human plasma. Controls consisted of spiked plasma with no adsorbent present. Adsorbents were incubated at 37° C. while shaking (90 rpm). At 5, 30 and 60 min time points, samples were centrifuged (125 g) and the supernatant collected and stored at −20° C. prior to ELISA (BD Biosciences) analysis for the presence of cytokines. Samples were diluted 1:4 (TNF-α, IL-8, IL-1β) and 1:10 (IL-6) in assay diluent prior to analysis.

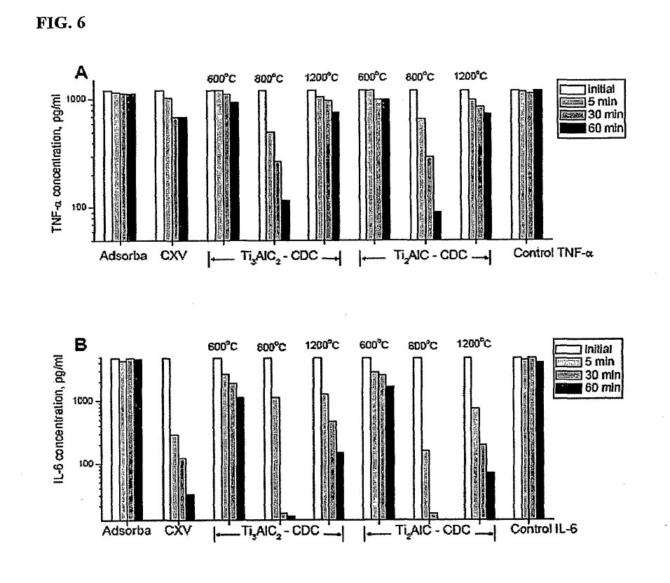

FIG. 6 compares efficiency of removal of two selected cytokines (tumor necrosis factor alpha (TNF-α) and interleukin-6 (IL-6)) from human plasma using the investigated carbons. Adsorption of TNF-α is known to be the most challenging task, probably due to a large size (>9.4 nm) of the trimeric (most common) form of this cytokine Reed C et al. Protein Engineering. 1997 October; 10(10):1101-7. Adsorba 300C and CDC produced at 600° C., which have small pores, did not noticeably change the protein concentration over time. CDC produced at 1200° C. and CXV also demonstrated a limited success in the adsorption of TNF-α, decreasing its concentration by about 40% after one hour of adsorption (FIG. 6A), similar to that observed in advanced porous carbon hemoadsorption systems Kellum J A et al. Critical Care Medicine. 2004 March; 32(3):801-5. In contrast, both CDC samples prepared at 800° C. decreased the protein concentration by over 13 times in this time period. Thus, in these experiments CDCs outperformed any other previously known material or method for the efficient removal of TNF-α, and the results are comparable only to highly specific antibody-antigen interactions. Weber V et al. Biomacromolecules. 2005 July-August; 6(4):1864-70; Hinterdorfer P et al. Proc. Nat. Acad. Sci. USA. 1996 Apr. 16; 93(8):3477-81.

Adsorption of the smaller cytokine IL-6 by most of the studied carbons was noticeably higher, but demonstrated similar trends (FIG. 6B). Strictly microporous Adsorba 300C was clearly inefficient. However, CDCs prepared at 600° C., having a limited amount of mesopores (pores having characteristic dimensions in the range of 2 to about 50 nm), adsorbed 66 to 77% of the cytokines initially present in the solution in one hour. The CDCs produced at 1200° C. demonstrated 97-98.5% adsorption, which is comparable to the CXV sample, capable of adsorbing ˜99%. The CDCs prepared from Ti₂AlC at 800° C., having the most developed mesoporosity decreased IL-6 concentration by ˜99.8%; the remaining IL-6 was close to the detection limit of the ELISA assay used.

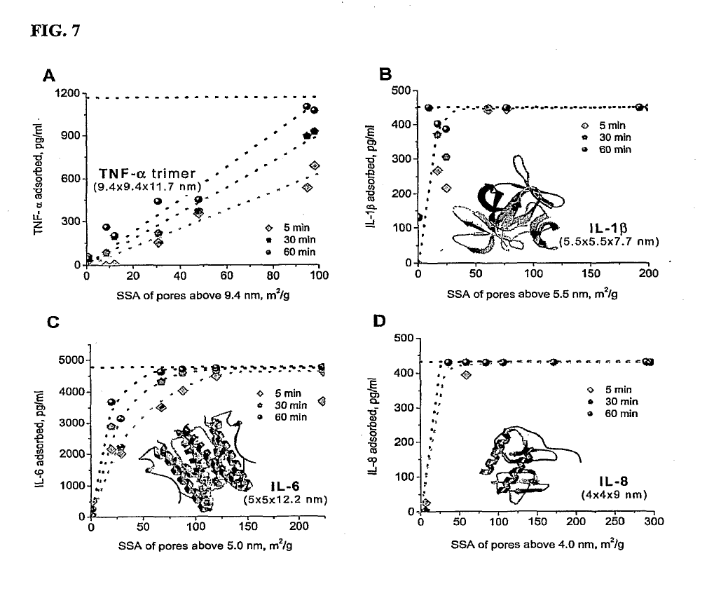

A clear dependence of protein removal efficiency on the PSDs of the porous carbons is seen when protein adsorption is plotted as a function of the carbons' accessible surface area, which is approximated as the SSA of pores exceeding the smallest protein dimension in size (FIG. 7). Dimensions of the investigated cytokines were considered to be: 9.4×9.4×11.7 nm (trimer of TNF-α) (Reed C et al. Protein Engineering. 1997 October; 10(10):1101-7), 5.5×5.5×7.7 nm (IL-1β) (Einspahr H et al. J Cryst Growth. 1988; 90(1-3):180-7), 5×5×12.2 nm (IL-6) (Somers W et al. Embo Journal. 1997 Mar. 3; 16(5):989-97), 4×4×9 nm (IL-8) (Rajarathnam K et al. Biochemistry. 1995 Oct. 10; 34(40):12983-90). The larger the surface areas of the porous carbons accessible to a given cytokine (“SSA_{acc}”), the more cytokines were adsorbed at a given time (FIGS. 7A, 7B, 7C, 7D). Some scattering in the results obtained could be explained by experimental errors in the estimation of the cytokine concentration and the carbon PSD. Depending on the cytokine and its initial concentration, values of the SSA_{acc} above 50-100 m²/g were generally sufficient for fast and efficient cytokine removal. The relatively short and small IL-1β and IL-8 cytokines diffused so rapidly into the carbon pores that 5 min was sufficient to adsorb most of these proteins by carbons with SSA_{acc} exceeding 50 m²/g (FIGS. 7B & 7D). The existence of larger channels within these carbons should have further accelerated the adsorption process. IL-6 demonstrated slower adsorption (FIG. 7C), probably due to its longer dimensions and hence slower diffusion within the carbon pore structure. The TNF-α trimer, the largest adsorbate, demonstrated a further decrease in adsorption rate (FIG. 7A) as the amount of pores, exceeding three times the adsorbate size needed for fast diffusion, was limited (FIGS. 3C & 3G).

Historically, in medical sciences and applied medicine, activated carbons are considered to be high SSA carbons of ultra purity. Differentiation of activated carbons with respect to difference in their PSD is uncommon. In fact, the same carbon materials are often used for adsorption of various species, from gases to organic molecules. However, since most commercial medical grade activated carbons, including Adsorba, are primarily microporous (FIGS. 3A, 4A), adsorption of inflammatory mediators with size exceeding 2 nm could only take place on the particles' surface (FIG. 1A). The calculated SSA of spherical carbon particles with a 10 μm diameter and 50% porosity is only ˜0.3 m²/g, which is much smaller than the 386-406 m²/g SSA of mesopores (2-50 nm) in the CDCs produced at 800° C. It is thus not too surprising that clinical trials of commercial extracorporeal adsorption systems did not show significantly decreased mortality in patients with sepsis. Reinhart K et al. Critical Care Medicine. 2004 August; 32(8):1662-8; Cole L et al. Critical Care Medicine. 2002 January; 30(1):100-6. Large biological molecules can move through pores of appropriate size—translocation of DNA through a nanotube were recently demonstrated (Fan R et al. Nano Letters. 2005 September; 5(9):1633-7)—and can be adsorbed in the bulk of the adsorbent particles (FIG. 1B). Pore size control is thus a key issue for achieving highly-efficient removal of large cytokines from blood plasma. The present invention demonstrates that engineering of novel nanostructured carbon adsorbents with rationally optimized porosity provide a solution for adsorption systems and can be used for any purpose in which the removal of particles from a fluid in a desired end result. For example, the present compositions, systems, and methods can be used in the treatment of individuals suffering from severe sepsis or any other inflammatory response. Similar approaches can be used for the selective adsorption of other large organic molecules (including viruses) for other medical or non-medical applications.

The disclosures of each patent, patent application and publication cited or described in this document are hereby incorporated herein by reference, in their entirety.

Those skilled in the art will appreciate that numerous changes and modifications can be made to the preferred embodiments of the invention and that such changes and modifications can be made without departing from the spirit of the invention. It is, therefore, intended that the appended claims cover all such equivalent variations as fall within the true spirit and scope of the invention.

1-50. (canceled)

51. An adsorption system comprising a carbon composition produced from a carbon-containing inorganic precursor comprising a plurality of pores, a plurality of said pores having characteristic dimensions from about 4 to about 50, wherein said composition adsorbs one or more particles from a fluid.

52. The adsorption system of claim 51 wherein said carbon-containing inorganic precursor comprises carbide.

53. The adsorption system of claim 52 wherein said carbide comprises ternary carbide or ternary carbonitride.

54. The adsorption system of claim 53 wherein said ternary carbide comprises a MAX phase group layered carbide.

55. The adsorption system of claim 51 wherein a substantial proportion of said pores are substantially slit-shaped.

56. The adsorption system of claim 51, said carbon composition having a total pore volume greater than 1.27 cc/g, as measured by N₂ adsorption at 77 K.

57. The adsorption system of claim 51, wherein the total volume of said pores having characteristic dimensions greater than about 4 nm is greater than 0.554 cc/g, as measured by N₂ adsorption at 77 K.

58. The adsorption system of claim 51, comprising a plurality of pores having characteristic dimensions greater than about 5 nm, wherein the total volume of said pores having characteristic dimensions greater than about 5 nm is greater than 0.434 cc/g, as measured by N₂ or Ar adsorption and analyzed according to the Brunauer-Emmet-Teller method.

59. The adsorption system of claim 51, comprising a plurality of pores having characteristic dimensions greater than about 5.5 nm, wherein the total volume of said pores having characteristic dimensions greater than about 5.5 nm is greater than 0.377 cc/g, as measured by N₂ or Ar adsorption and analyzed according to the non-local density functional theory method.

60. The adsorption system of claim 51, comprising a plurality of pores having characteristic dimensions greater than about 9.5 nm, wherein the total volume of said pores having characteristic dimensions greater than about 9.5 nm is greater than 0.0824 cc/g, as measured by N₂ adsorption at 77 K.

61. The adsorption system of claim 51 having a total specific surface area greater than 1652 m²/g, as measured according to the Brunauer-Emmet-Teller method.

62. The adsorption system of claim 51 having a total specific surface area greater than 1362 m²/g, as measured according to the non-local density functional theory method.

63. The adsorption system of claim 51, wherein the specific surface area of pores having characteristic dimensions greater than 4 nm is greater than 172 m²/g, as measured by N₂ adsorption at 77 K.

64. The adsorption system of claim 51 comprising particles of Ti₂AlC reacted with chlorine.

65. The adsorption system of claim 64 comprising particles of Ti₂AlC reacted with chlorine at a temperature at or exceeding about 600° C.

66. The adsorption system of claim 64 comprising particles of Ti₂AlC reacted with chlorine at a temperature at or exceeding about 800° C.

67. The adsorption system of claim 64 comprising particles of Ti₂AlC reacted with chlorine at a temperature at or exceeding about 1200° C.

68. The adsorption system of claim 51 comprising particles of Ti₃AlC₂ reacted with chlorine.

69. The adsorption system of claim 68 comprising particles of Ti₃AlC₂ reacted with chlorine at a temperature at or exceeding about 600° C.

70. The adsorption system of claim 68 comprising particles of Ti₃AlC₂ reacted with chlorine at a temperature at or exceeding about 800° C.

71. The adsorption system of claim 68 comprising particles of Ti₃AlC₂ reacted with chlorine at a temperature at or exceeding about 1200° C.

72. The adsorption system of claim 51 having a N₂ sorption profile of at least 1000 cc/g N₂ at 1.0 P/Pₒ.

73. The adsorption system of claim 51, wherein said composition adsorbs one or more proteins from a fluid.

74. The adsorption system of 73, wherein at least one of said proteins is an inflammatory mediator.

75. The adsorption system of claim 74, wherein said inflammatory mediator is a cytokine.

76. The adsorption system of claim 75, wherein said composition adsorbs TNF-α cytokine from a fluid.

77. The adsorption system of claim 76, wherein said composition adsorbs at least about 40% of said TNF-α in about 60 min.

78. The adsorption system of claim 76, wherein said composition adsorbs at least about 60% of said TNF-α in about 60 min.

79. The adsorption system of claim 76, wherein said composition adsorbs at least about 80% of said TNF-α in about 60 min.

80. The adsorption system of claim 75, wherein said composition adsorbs IL-6 cytokine from a fluid.

81. The adsorption system of claim 80, wherein said composition adsorbs at least about 50% of said IL-6 cytokine in about 60 min.

82. The adsorption system of claim 80, wherein said composition adsorbs at least about 70% of said IL-6 cytokine in about 60 min.

83. The adsorption system of claim 80, wherein said composition adsorbs at least about 90% of said IL-6 cytokine in about 60 min.

84. The adsorption system of claim 51 comprising a plurality of pores having characteristic dimensions greater than 5 nm, said pores having a total specific surface area greater than 120 m²/g, as measured by N₂ adsorption at 77 K.

85. The adsorption system of claim 51 comprising a plurality of pores having characteristic dimensions greater than 5.5 nm, said pores having a total specific surface area greater than 98.3 m²/g, as measured by N₂ adsorption at 77 K.

86. The adsorption system of claim 51 comprising a plurality of pores having characteristic dimensions greater than 9.5 nm, said pores having a total specific surface area greater than 14.6 m²/g, as measured by N₂ adsorption at 77 K.

87. The adsorption system of claim 51, at least about 30 volumetric percentage of said pores, as measured by N₂ adsorption at 77 K, having characteristic dimensions equal to or greater than about 9.5 nm, wherein said carbon composition adsorbs TNF-α from a fluid.

88. The adsorption system of claim 87, wherein at least about 50 volumetric percentage of said pores, as measured by N₂ adsorption at 77 K, have characteristic dimensions equal to or greater than about 9.5 nm.

89. The adsorption system of claim 87, wherein at least about 70 volumetric percentage of said pores, as measured by N₂ adsorption at 77 K, have characteristic dimensions equal to or greater than about 9.5 nm.

90. The adsorption system of claim 51, at least about 30 volumetric percentage of said pores, as measured by N₂ adsorption at 77 K, having characteristic dimensions greater than about 5.5 nm, wherein said carbon composition adsorbs IL-1β from a fluid.

91. The adsorption system of claim 90, wherein at least about 50 volumetric percentage of said pores, as measured by N2 adsorption at 77 K, have characteristic dimensions greater than about 5.5 nm.

92. The adsorption system of claim 90, wherein at least about 70 volumetric percentage of said pores, as measured by N₂ adsorption at 77 K, have characteristic dimensions greater than about 5.5 nm.

93. The adsorption system of claim 51, at least about 30 volumetric percentage of said pores, as measured by N₂ adsorption at 77 K, having characteristic dimensions greater than about 5 nm, wherein said carbon composition adsorbs IL-6 from a fluid.

94. The adsorption system of claim 93, wherein at least about 50 volumetric percentage of said pores, as measured by N₂ adsorption at 77 K, have characteristic dimensions greater than about 5 nm.

95. The adsorption system of claim 93, wherein at least about 70 volumetric percentage of said pores, as measured by N₂ adsorption at 77 K, have characteristic dimensions greater than about 5 nm.

96. The adsorption system of claim 51, at least about 30 volumetric percentage of said pores, as measured by N₂ adsorption at 77 K, having characteristic dimensions greater than about 4 nm, and said carbon composition adsorbs IL-8 from a fluid.

97. The adsorption system of claim 96, wherein at least about 50 volumetric percentage of said pores, as measured by N₂ adsorption at 77 K, have characteristic dimensions greater than about 4 nm.

98. The adsorption system of claim 96, wherein at least about 70 volumetric percentage of said pores, as measured by N₂ adsorption at 77 K, have characteristic dimensions greater than about 4 nm.

99. The adsorption system according to claim 51, further comprising a binder.

100. The adsorption system according to claim 99, wherein said binder is a polymer.

101-173. (canceled)
